(19) United States
(12) Patent Application Publication
(10) Pub. No.: US 2022/0395600 A1
(43) Pub. Date: Dec. 15, 2022

(54) WARMER FOR SCENTED CANDLE

(71) Applicant: Chan Soo KIM, Seoul (KR)

(72) Inventor: Chan Soo KIM, Seoul (KR)

(21) Appl. No.: 17/346,129

(22) Filed: Jun. 11, 2021

(51) Int. Cl. A61L 9/03; F21V 35/00

(52) U.S. Cl. CPC A61L 9/03; F21V 35/00; A61L 2209/12; A61L 2209/13

(57) ABSTRACT

Disclosed is a scented candle warmer for overcoming trouble and inconvenience at once by easily processing and storing melted drippings and then simply discarding the drippings, and including a main body part including an accommodation groove for accommodating a collection container therein and a luminaire part formed on an upper part thereof, the collection container having a storage space for storing drippings of a scented candle and accommodated on or removed from the accommodation groove of the main body part, and a support part configured to detachably cover an upper part of the collection container and to simultaneously accommodate a scented candle container on an upper part thereof.

### FIELD OF INVENTION

The present invention relates to a scented candle warmer, and more particularly to a scented candle warmer for overcoming trouble and inconvenience at once by easily processing and storing melted drippings and then simply discarding the drippings.

### BACKGROUND OF INVENTION

As is well known, a scented candle warmer is a device that irradiates light at high temperature to an upper part of a scented candle to gradually melt the upper part of the scented candle and to emit scent of the scented candle to the air according to a melting phenomenon.

Referring to FIG. 1, such a conventional scented candle warmer is configured to place a scented candle container 20 containing a solid scented candle 21 on an upper part of a base part 10 and to emit light toward an upper part of the candle container 20 from a luminaire 12 through a column support 11.

Thus, when the luminaire 12 emits light, scent is released into the air while the upper part of the scented candle 21 is gradually melted.

Then, when a scent ingredient included in a melted part of the scented candle is entirely volatilized, scent is not released any longer, and thus liquid (drippings) of the scented candle 21 melted on the upper part of the scented candle container 20 may be discarded, and the scented candle 21 may be accommodated on the base part 10 again to allow the luminaire 12 to emit light to the scented candle 21.

In order to use the scented candle warmer, this procedure may be repeatedly performed.

However, in reality, when a scented candle warmer is used, since there is no place to dump drippings dissolved in the scented candle container 20, the drippings need to be dumped in a trash can, and accordingly, there is a problem in that it is cumbersome to bring and move the scented candle container 20 and it is difficult to clean when the drippings are hardened within the trash can, and also, there is a problem in terms of safety due to hot drippings.

For reference, drippings should not be thrown away in a drain.

### CITED REFERENCE

### Patent Document

Korean Patent Publication No. 10-2051610 (Registered on Nov. 27, 2019)
Korean Patent Publication No. 10-2103434 (Registered on Apr. 16, 2020)
Korean Patent Publication No. 10-1973325 (Registered on Apr. 22, 2019)
Korean Patent Laid-Open Publication No. 10-2020-0007514 (Published on Jan. 22, 2020)

### Technical Solution

Therefore, the present invention provides a scented candle warmer for overcoming trouble and inconvenience at once by easily processing and storing melted drippings and then simply discarding the drippings.

### Technical Goal of the Invention

To achieve the technical goal, the present invention provides a scented candle warmer including a main body part including an accommodation groove for accommodating a collection container therein and a luminaire part formed on an upper part thereof, the collection container having a storage space for storing drippings of a scented candle and accommodated on or removed from the accommodation groove of the main body part, and a support part configured to detachably cover an upper part of the collection container and to simultaneously accommodate a scented candle container on an upper part thereof.

A protrusion inserted into a central groove part of the collection container may be formed at a center of a bottom surface of the accommodation groove of the main body part.

An upper part and a lower part of the main body part may be detachably installed and are coupled to each other through couplers formed at opposite ends of the lower part, an insertion hole into which an accommodation part of the lower part is inserted may be formed in the upper part, and the lower part may include an accommodation groove in which the collection container is accommodated.

A central groove part into which a protrusion of the main body part is inserted may be formed at a center of a bottom surface of the collection container.

The support part may include an accommodation part for stably accommodating and supporting a lower surface of the scented candle container through a flange part of an edge, and an edge part extending in a downward direction of the flange part and installed to cover an upper part thereof by the collection container.

Handle holes for moving the support part with fingers may be formed in the accommodation part of the support part.

The present invention provides a scented candle warmer including a main body part including a luminaire part installed thereon through a column support and including a collection container placed thereon, the collection container being placed on an upper part of the base part and having a storage space for storing drippings of a scented candle, and a support part configured to detachably cover an upper part of the collection container and to simultaneously accommodate a scented candle container on an upper part thereof.

The support part may include an accommodation part for stably accommodating and supporting a lower surface of the scented candle container through a flange part of an edge, and an edge part extending in a downward direction of the flange part and installed to cover an upper part thereof by the collection container.

The base part may be a space in which the collection container is accommodated and may include a step part and an accommodation part.

The collection container may be formed of a flexible synthetic resin material.

### Effect of Invention

The present invention provides an advantage in that trouble or inconvenience in use is overcome by easily processing and storing melted drippings and then simply discarding the drippings.

### BRIEF DESCRIPTION OF DRAWINGS

FIG. 1 is a diagram showing the configuration of a general scented candle warmer.

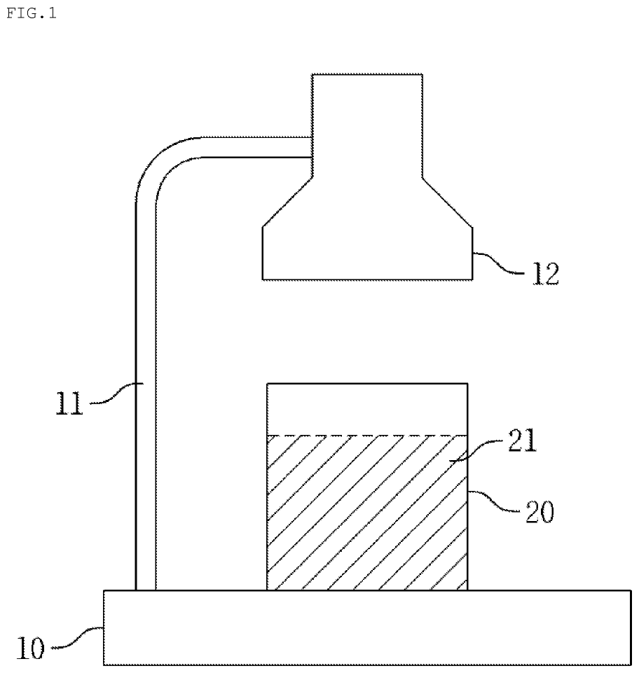

FIG. 2 is a diagram showing the configuration of a scented candle warmer according to the present invention.

FIG. 3 is a structure view showing the state in which a scented candle container is installed in FIG. 2.

FIG. 4 is an exploded perspective view of a scented candle warmer according to the present invention.

FIG. 5 is a perspective view of a bottom surface of a main body part according to the present invention.

FIG. 6 is a perspective view of a support part according to the present invention.

FIG. 7 is a diagram showing a state of use of a scented candle warmer according to the present invention.

FIG. 8 is an exploded perspective view of a main body part according to the present invention.

FIG. 9 is a diagram showing the configuration of a scented candle warmer according to another embodiment of the present invention.

FIG. 10 is a structure view showing the state in which the scented candle container, the support part, and the collection container are removed in FIG. 9.

### BEST MODE

Hereinafter, some embodiments of the present invention will be described in detail with reference to the accompanying drawings.

In the following description, like reference numerals designate like elements although the elements are shown in different drawings. Further, in the following description of the embodiments of the present invention, a detailed description of known functions and configurations incorporated herein will be omitted for clarity and brevity.

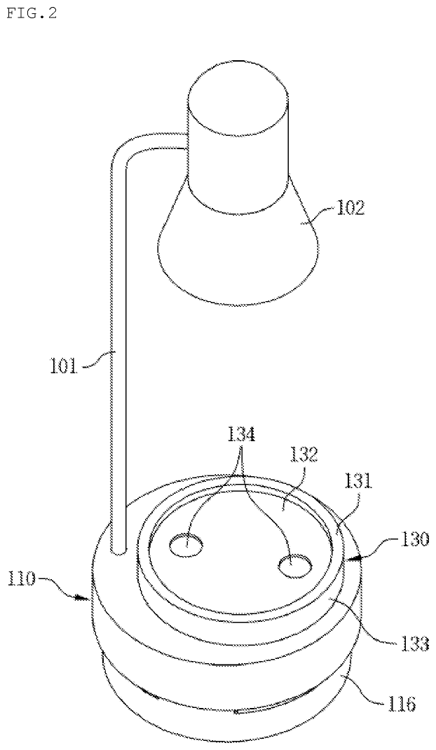

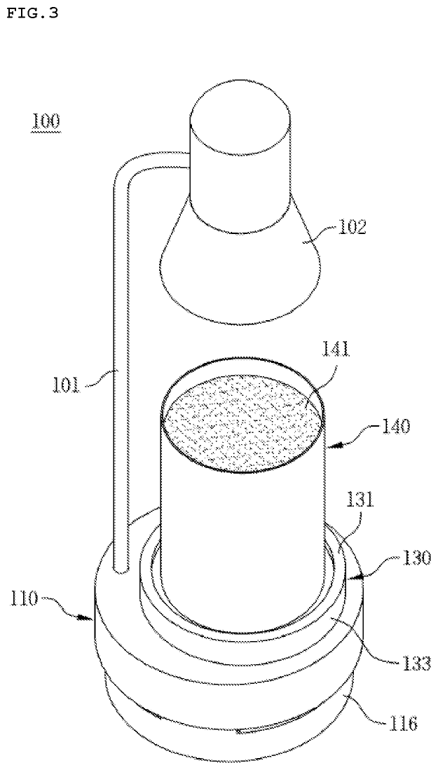

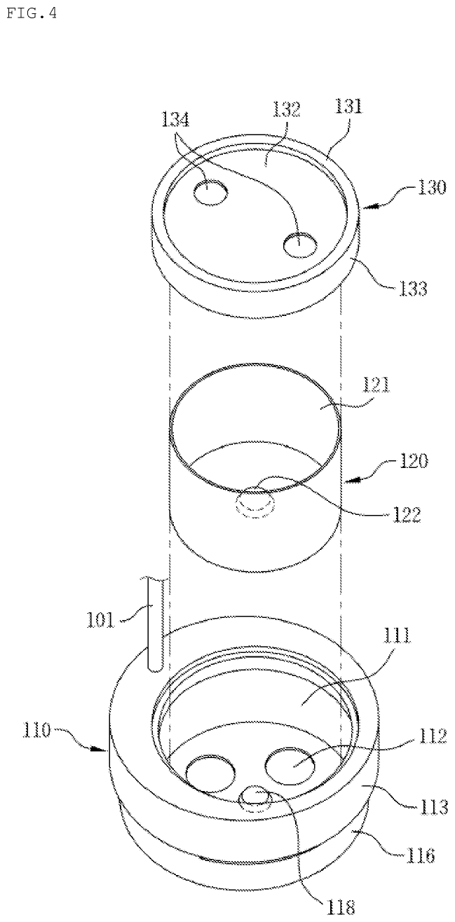

FIG. 2 is a diagram showing the configuration of a scented candle warmer according to the present invention. FIG. 3 is a structure view showing the state in which a scented candle container is installed in FIG. 2. FIG. 4 is an exploded perspective view of a scented candle warmer according to the present invention.

As shown in the drawings, a scented candle warmer 100 according to the present invention may include a main body part 110, a collection container 120, and a support part 130.

A luminaire part 102 may be installed at the main body part 110 through a column support 101.

The present invention is not limited to the case in which the luminaire part 102 is fixed through the column support 101 connected to the main body part 110 but may have any configuration as long as the luminaire part 102 emits light toward an upper part of a scented candle container 140 positioned below the luminaire part 102.

The main body part 110 may include an accommodation groove 111 in which the collection container 120 is accommodated.

A protrusion 118 may be formed at the center of a bottom surface of the accommodation groove 111.

The protrusion 118 may be stably supported in the accommodation groove 111 to be inserted into a central groove part 122 of the accommodated collection container 120.

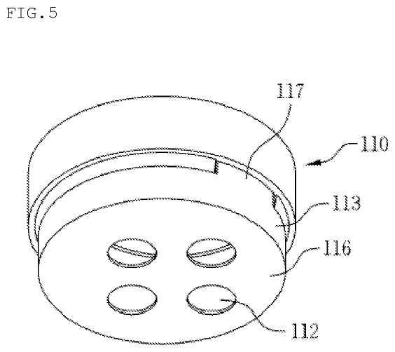

As shown in FIG. 5, a plurality of through holes 112 may be formed in the bottom surface of the accommodation groove 111.

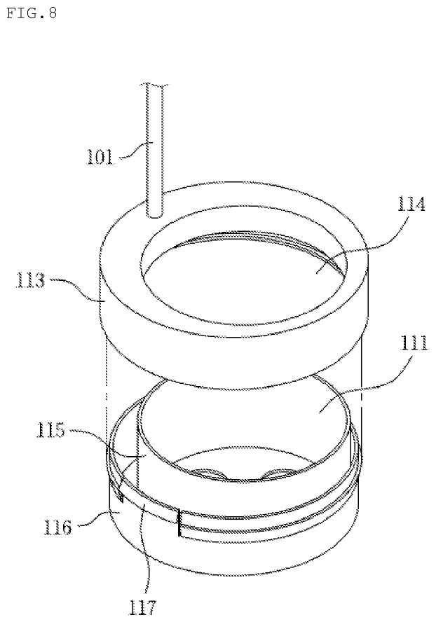

As shown in FIG. 8, the main body part 110 may be configured in such a way that an upper part 113 and a lower part 116 are detachably installed and are coupled to each other through couplers 117 formed at opposite ends of the lower part 116, an insertion hole 114 into which an accommodation part 115 of the lower part 116 is inserted is formed in the upper part 113, and the lower part 116 includes the accommodation part 115 for forming the accommodation groove 111 in which the collection container 120 is to be accommodated.

The collection container 120 may store drippings of the scented candle, may include a storage space 121, and may be configured to be accommodated on or removed from the accommodation groove 111 of the main body part 110.

The central groove part 122 into which the protrusion 118 of the main body part 110 is inserted may be formed at the center of a bottom surface of the collection container 120, and thus when the collection container 120 is accommodated in the accommodation groove 111 of the main body part 110, the collection container 120 may be stably supported by inserting the protrusion 118 into the central groove part 122.

The collection container 120 may be a consumable that is discarded when collecting a predetermined amount of scented candle drippings.

The collection container 120 may be formed of a flexible synthetic resin material.

The flexible synthetic resin material may be silicon, and the collection container 120 formed of silicon may be reused after only hardened drippings are easily removed using elasticity unique to a silicone material when drippings stored in the collection container 120 is hardened.

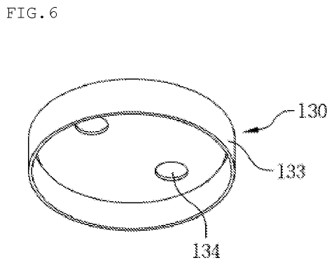

As shown in FIGS. 4 and 6, the support part 130 may cover an upper part of the collection container 120 and may simultaneously accommodate the scented candle container 140 on the support part 130, and may include an accommodation part 132 for stably accommodating and supporting a lower surface of the scented candle container 140 through a flange part 131 of an edge, and an edge part 133 extending in a downward direction of the flange part 131 and installed to cover an upper part thereof by the collection container 120.

Handle holes 134 for moving the support part 130 with fingers may be formed in the accommodation part 132 of the support part 130.

An assembly procedure, an operation, and an effect of the scented candle warmer 100 according to the present invention as configured above will be described.

First, the collection container 120 may be installed into the accommodation groove 111 of the main body part 110 in which the luminaire part 102 is installed through the column support 101.

When the collection container 120 is inserted into the accommodation groove 111, if the central groove part 122 of the collection container 120 is accommodated on the protrusion 118 formed on the bottom surface of the accommodation groove 111, the collection container 120 may be stably supported without shaking.

As such, when the collection container 120 is installed on the main body part 110, the upper part of the collection container 120 may be covered by the support part 130.

Such a state may become in the state shown in FIG. 2, and then, the scented candle container 140 may be placed on the upper part of the support part 130, as shown in FIG. 3.

In this case, a lower part of the scented candle container 140 may be accommodated on the accommodation part 132 of the support part 130.

Then, when the luminaire part 102 is turned on, light may be emitted to the upper part of a scented candle 141 of the scented candle container 140 while a lamp (e.g., a halogen lamp) is turned on, and the solid scented candle 141 may be gradually melted by heat of the emitted light.

When the scented candle 141 is melted, scent may be released, and the scented candle 141 may perform a function of the scented candle.

Then, when the scented candle 141 melts to some extent, scent stops being released, and thus the time at which the melted drippings needs to be discarded may be reached.

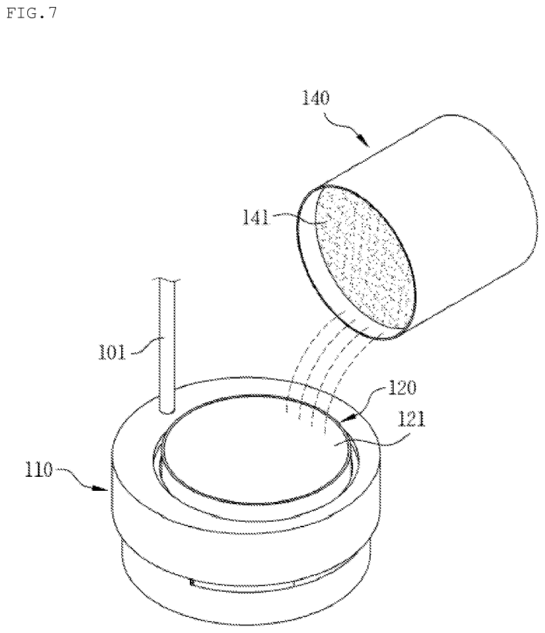

Thus, when the time at which drippings need to be discarded is reached, the scented candle container 140 may be moved, the support part 130 may be taken off from the collection container 120 using fingers in finger holes 134, and then drippings on the upper part of the scented candle container 140 may be poured into the collection container 120, as shown in FIG. 7.

Then, the support part 130 may be covered on the upper part of the collection container 120 again, and the scented candle container 140 may be placed on the collection container 120.

Thus, the upper part of the scented candle 141 may perform a function of a scented candle while being gradually melted by the luminaire part 102 again.

That is, according to the present invention, drippings melted in the scented candle container 140 may be immediately collected and stored in the collection container 120 installed in the main body part 110, and then, when a predetermined amount of drippings are stored, the scented candle container 140 may be simply discarded and processed, which is advantageous.

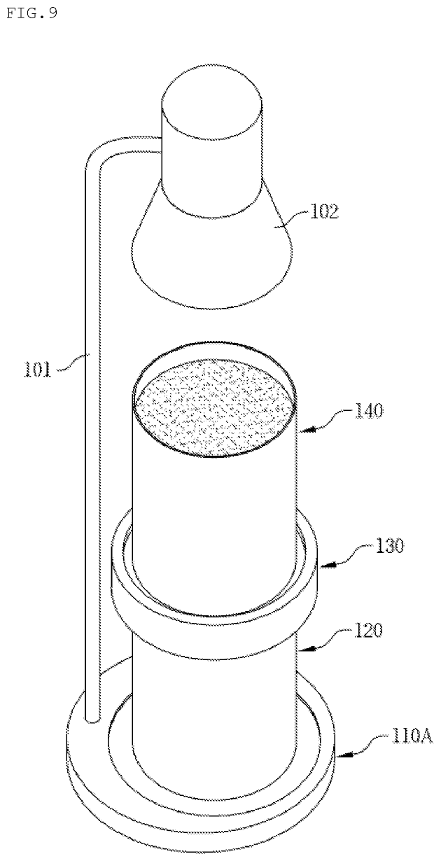

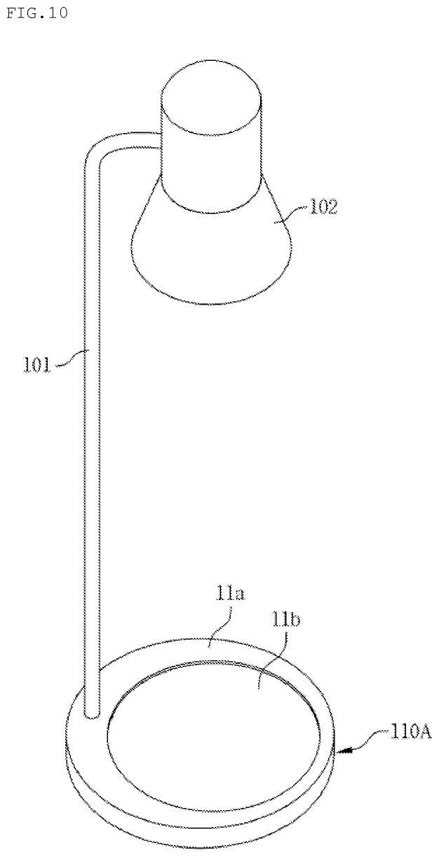

FIG. 9 is a diagram showing the configuration of a scented candle warmer according to another embodiment of the present invention. FIG. 10 is a structure view showing the state in which the scented candle container, the support part, and the collection container are removed in FIG. 9.

The scented candle warmer according to another embodiment of the present invention may include a base part 110A on which the luminaire part 102 is installed through the column support 101 and the collection container 120 is placed, the collection container 120 placed on an upper part of the base part 110A and having the storage space 121, and the support part 130 that covers the upper part of the collection container 120 and simultaneously accommodates the scented candle container 140 on the upper part thereof and includes the accommodation part 132 for stably accommodating and supporting the lower surface of the scented candle container 140 through an edge thereof, and the edge part 133 that extends in a downward direction of the flange part 131 and is installed to cover an upper part thereof by the collection container 120.

The base part 110A may be a space in which the collection container 120 is accommodated and may include a step part 11a and an accommodation part 11b.

The scented candle warmer according to another embodiment of the present invention as configured above may have the same effect as the aforementioned operation.

While the present invention has been particularly shown and described with reference to exemplary embodiments thereof, it will be understood by those of ordinary skill in the art that various changes in form and details may be made therein without departing from the spirit and scope of the present invention as defined by the following claims.

[Description of reference numerals]

| | | | |
|---|---|---|---|
| 100: | scented candle warmer | 101: | column support |
| 102: | luminaire part | 110: | main body part |
| 111: | accommodation groove | 112: | through hole |
| 113: | upper part | 114: | insertion hole |
| 115: | accommodation part | 116: | lower part |
| 120: | collection container | 121: | storage space |
| 130: | support part | 131: | flange part |
| 132: | accommodation part | 133: | edge part |
| 134: | finger hole | 140: | scented candle container |
| 141: | scented candle | | |

1. A scented candle warmer comprising:
a main body part including an accommodation groove for accommodating a collection container therein and a luminaire part formed on an upper part thereof;
the collection container having a storage space for storing drippings of a scented candle and accommodated on or removed from the accommodation groove of the main body part; and
a support part configured to detachably cover an upper part of the collection container and to simultaneously accommodate a scented candle container on an upper part thereof.

2. The scented candle warmer according to claim 1, wherein a protrusion inserted into a central groove part of the collection container is formed at a center of a bottom surface of the accommodation groove of the main body part.

3. The scented candle warmer according to claim 1, wherein an upper part and a lower part of the main body part are detachably installed and are coupled to each other through couplers formed at opposite ends of the lower part, an insertion hole into which an accommodation part of the lower part is inserted is formed in the upper part, and the lower part includes an accommodation groove in which the collection container is accommodated.

4. The scented candle warmer according to claim 1, wherein a central groove part into which a protrusion of the main body part is inserted is formed at a center of a bottom surface of the collection container.

5. The scented candle warmer according to claim 1, wherein the collection container is formed of a flexible synthetic resin material.

6. The scented candle warmer according to claim 1, wherein the support part includes:
an accommodation part for stably accommodating and supporting a lower surface of the scented candle container through a flange part of an edge; and
an edge part extending in a downward direction of the flange part and installed to cover an upper part thereof by the collection container.

7. The scented candle warmer according to claim 1, wherein handle holes for moving the support part with fingers are formed in the accommodation part of the support part.

8. A scented candle warmer comprising:
a main body part including a luminaire part installed thereon through a column support and including a collection container placed thereon;
the collection container being placed on an upper part of the base part and having a storage space for storing drippings of a scented candle; and
a support part configured to detachably cover an upper part of the collection container and to simultaneously accommodate a scented candle container on an upper part thereof.

9. The scented candle warmer according to claim 8, wherein the support part includes:
an accommodation part for stably accommodating and supporting a lower surface of the scented candle container through a flange part of an edge; and
an edge part extending in a downward direction of the flange part and installed to cover an upper part thereof by the collection container.

10. The scented candle warmer according to claim 8, wherein the base part is a space in which the collection container is accommodated and includes a step part and an accommodation part.

11. The scented candle warmer according to claim 8, wherein the collection container is formed of a flexible synthetic resin material.
